SEQUENCE LISTING (23 sequences)

SEQ ID NO: 1 - DNA, 61 bp, Artificial Sequence
  Selected DNA ligand (OTA1.12)
tggtggctgtaggtcagcatctgatcgggtgtgggtggcgtaaagggagcatcggacaacg

SEQ ID NO: 2 - DNA, 49 bp, Artificial Sequence
  Selected DNA ligand (OTA1.13)
tggtggctgtaggtcaggggtgaaacgggtcccggagcatcggacaacg

SEQ ID NO: 3 - DNA, 61 bp, Artificial Sequence
  Selected DNA ligand (OTA1.14)
tggtggctgtaggtcagcagtcctagatcgggtgtggctggcttgggagcatcggacaacg

SEQ ID NO: 4 - DNA, 61 bp, Artificial Sequence
  Selected DNA ligand (OTA2.2)
tggtggctgtaggtcaactgtccgtcgggtttagggtggcgttcgggagcatcggacaacg

SEQ ID NO: 5 - DNA, 61 bp, Artificial Sequence
  Selected DNA ligand (OTA2.3)
tggtggctgtaggtcatcagtcccgatcaggtgtgggtggcattgggagcatcggacaacg

SEQ ID NO: 6 - DNA, 61 bp, Artificial Sequence
  Selected DNA ligand (OTA2.4)
tggtggctgtaggtcaccaaatgcgacggggcctgttttaatgggggagcatcggacaacg

SEQ ID NO: 7 - DNA, 60 bp, Artificial Sequence
  Selected DNA ligand (OTA2.6)
tggtggctgtaggtcacgtacggtgggaacggttcctcttagggtgagcatcggacaacg

SEQ ID NO: 8 - DNA, 61 bp, Artificial Sequence
  Selected DNA ligand (OTA2.9)
tggtggctgtaggtcacaggtggcagatcgggtgtgggtggcctgggagcatcggacaacg

SEQ ID NO: 9 - DNA, 61 bp, Artificial Sequence
  Selected DNA ligand (OTA2.10)
tggtggctgtaggtcaacatgcgactgaggctcggtttattgaggggagcatcggacaacg

SEQ ID NO: 10 - DNA, 61 bp, Artificial Sequence
  Selected DNA ligand (OTA2.11)
tggtggctgtaggtcacctgacgatcgggtgtgggttggcttgagggagcatcggacaacg

SEQ ID NO: 11 - DNA, 61 bp, Artificial Sequence
  Selected DNA ligand (OTA2.12)
tggtggctgtaggtcaccttgtagatcgggtgtggtttggcgtagggagcatcggacaacg

SEQ ID NO: 12 - DNA, 61 bp, Artificial Sequence
  Selected DNA ligand (OTA2.13)
tggtggctgtaggtcagcagtacgatcgggggtgggtggatgtagggagcatcggacaacg

SEQ ID NO: 13 - DNA, 14 bp, Artificial Sequence
  Consensus motif
gatcgggtgtgggt

SEQ ID NO: 14 - DNA, 16 bp, Artificial Sequence
  Forward primer
tggtggctgtaggtca

SEQ ID NO: 15 - DNA, 15 bp, Artificial Sequence
  Reverse primer
cgttgtccgatgctc

SEQ ID NO: 16 - DNA, 29 bp, Artificial Sequence
  Sulforhodamine DNA ligand
atgaccaagggtgggagggagggggtcat

SEQ ID NO: 17 - DNA, 18 bp, Artificial Sequence
  OTA1.13S
ggggtgaaacgggtcccg

SEQ ID NO: 18 - DNA, 41 bp, Artificial Sequence
  DNA ligand (OTA1.12.1)
gatcgggtgtgggtggcgtaaagggagcatcggacaacgat

SEQ ID NO: 19 - DNA, 36 bp, Artificial Sequence
  DNA ligand (OTA1.12.2)
gatcgggtgtgggtggcgtaaagggagcatcggaca

SEQ ID NO: 20 - DNA, 41 bp, Artificial Sequence
  DNA ligand (OTA1.12.3)
gatcgggtgtcggtggcgtaaagggagcatcggacaacgat

SEQ ID NO: 21 - DNA, 41 bp, Artificial Sequence
  DNA ligand (OTA1.12.4)
gatcgtgtgtcggtggcgtaaagggagcatcggacaacgat

SEQ ID NO: 22 - DNA, 39 bp, Artificial Sequence
  DNA ligand (OTA1.12.5)
gatcgggtgtgggtggcgtaaagggagcatcggacaacg

SEQ ID NO: 23 - DNA, 39 bp, Artificial Sequence
  DNA ligand (OTA1.12.6)
gatcgggtgtcggtggcgtaaagggagcatcggacaacg

(19) United States
(12) Patent Application Publication
(10) Pub. No.: US 2011/0306508 A1
(43) Pub. Date: Dec. 15, 2011

(54) Method of Mycotoxin Detection

(75) Inventors: Gregory Allen Penner, London (CA); Jorge Andres Cruz-Aguado, London (CA)

(73) Assignee: NeoVentures Biotechnology Inc.

(21) Appl. No.: 12/812,336

(22) PCT Filed: Jan. 9, 2009
(86) PCT No.: PCT/CA2009/000005

§ 371 Date: Oct. 27, 2010

Related U.S. Application Data
(60) Provisional application No. 61/020,365, filed on Jan. 10, 2008.

(51) Int. Cl. G01N 33/53; G01N 21/64; G01N 30/02; G01N 27/00; C40B 30/04; C12N 9/96; C12Q 1/68; C07K 1/14; C22C 24/00; C22C 28/00; C22C 14/00; C22C 27/02; C22C 27/06; C22C 22/00; C22C 38/00; C22C 19/07; C22C 19/03; C22C 9/00; C22C 18/00; C07H 21/04

(52) U.S. Cl. 506/9; 536/23.74; 436/501; 435/188; 435/6.19; 530/409; 420/400; 420/415; 420/416; 420/417; 420/424; 420/428; 420/434; 420/8; 420/435; 420/441; 420/469; 420/513

(57) ABSTRACT

The presence of mycotoxins in agricultural products necessitates large scale testing of a wide range of sample material to ensure the safety of food and feed. The mycotoxin ochratoxin A represents an enablement for all mycotoxins as the level of sensitivity necessary for regulatory requirements for this compound at the part per billion level are as low or lower than any other mycotoxin. This invention describes the identification of a set of DNA ligands with sufficiently high binding affinity and specificity for ochratoxin A to enable an improvement over existing methods for the separation, concentration and quantitative determination of ochratoxin A in sample material.

### FIELD OF THE INVENTION

The invention relates to DNA oligonucleotides that bind to mycotoxins. More specifically, the invention is directed to DNA ligands that have an increased affinity for mycotoxins that are present in certain agricultural products and are associated with human or animal health risks. As such, these novel DNA ligands provide the basis for new methods of determining the presence and/or concentration of mycotoxins in samples of interest and for removing the mycotoxins from the sample of interest. In one aspect of the invention the mycotoxin is ochratoxin A.

### BACKGROUND OF THE INVENTION

Mycotoxins are toxins produced by fungi. Major groups of mycotoxins include aflatoxins, ochratoxin, trichothecenes (including deoxynivalenol, T2-toxin and zearelone), fumosins and patulin. One of the most toxic of these mycotoxins is ochratoxin A (OTA).

The chemical compound OTA is more fully described as L-phenylalanine N-[5-chloro-3,4-dihydro-8-hydroxy-3-methyl-1-oxo-1H2-benzopyran-7-yl]carbonyl-(R)-isocoumarin, (C20H18ClNO6) and is commonly referred to as ochratoxin A (OTA). The molecular weight of this compound is 403.82 (g/mol). OTA is a mycotoxin that is produced by several fungal strains of Aspergillus and Penicillum (van der Merwe et al., Nature (1965) 205:1112-1113). In animal models a wide variety of toxicological effects have been noted including nephrotoxicity and nephrocarcinogenicity, neurotoxicity and immunotoxicity (WHO Food Additives Series (2001) No. 47:281-415). The mode of action whereby OTA causes all these effects remains unclear. OTA has been shown to nick DNA and to be the primary causal agent of kidney cancer in mouse studies (Creppy et al., (1985) Toxicology Letters 28:29-35).

The International Agency for Research on Cancer (IARC) classified OTA as being a possible carcinogen in humans (Group 2B) (IARC Monographs on the Evaluations of Carcinogenic Risks to Humans, (1993) 56:489-521). The Joint FAO/WHO Expert Committee on Food Additives (JECFA), after evaluation of OTA nephrotoxicity, proposed a provisional tolerable weekly intake of 0.1 μg mycotoxin a provisional tolerable weekly intake (PTWI) of 0.1 ng/kg body mass (equivalent to 14 ng/kg body mass/day) (WHO, (1996) Food Additives Series 35:363-376). Achievement of this objective to restrict OTA intake in humans requires an understanding of the ways in which this mycotoxin is entering the human diet. Primary entry points in Europe have been demonstrated to be cereals, wine, spices, coffee and beer (Jørgensen, (2005) Food Additives & Contaminants, 22:26-30). Draft European levels for regulation of ochratoxin A levels in various foods ranges from 10 μg/kg in instant coffee and raisins to 0.5 μg/kg in baby food formulations.

U.S. Pat. No. 5,178,832 teaches a method for the concentration and detection of mycotoxins that is based on the binding of mycotoxins to clay minerals. This patent, however, does not demonstrate how to separate ochratoxin A from certain other mycotoxins such as deoxynivalenol; does not provide an estimate of the level of sensitivity achievable for ochratoxin A detection; does not demonstrate that the method described can be used to separate ochratoxin A from ochratoxin B, or other forms of ochratoxin. A key shortcoming of this technology is that the mineral/mycotoxin interaction is both weak in terms of binding capacity, and extremely limited in terms of ligand specificity.

U.S. Patent Application No. 20050100959 teaches the use of a flow through analytical device in combination with an antibody for ochratoxin A as a means of rapid detection. The concentration appears to be reproducible to a level of 4 ng of ochratoxin per gram of sample material. This method is reliant on the use of an antibody for ochratoxin A.

The use of antibodies to quantitatively determine the presence of mycotoxins in a wide variety of samples is known in the prior art (Rousseau et al., (1987) Applied and Environmental Microbiology, 53:514-518; Candlish, et al., (1986). Lett. Appl. Microbiol. 3:9-11). The monoclonal antibodies identified by these groups have strong binding affinities for OTA and are highly specific for this particular ligand. The sensitivity level achieved with the Rousseau et al., antibody was 0.2 ppb through the use of radio-labeled OTA and a competition assay The sensitivity and specificity of existing antibodies for OTA complies with emerging regulatory guidelines for OTA globally. There are commercial kits available based on the use of antibodies in immuno-affinity columns to separate and concentrate OTA from large samples, and for the direct determination of OTA concentration.

Antibodies however have several limitations in regard to use in rapid field ready diagnostic applications. Antibodies must be produced in biological systems. This limits the decrease in cost achieved increasing production amounts that is implicit with chemical synthesis. The biological production of antibodies also requires a higher level of quality assurance analysis than chemical synthesis.

The sample preparation required for OTA analysis present an array of difficulties for antibody based analysis. Efficient extraction of OTA from any sample matrix can only be achieved with high levels of organic solvents. Antibody structure is altered by exposure to organic solvents in ways that completely eliminate ligand binding. To overcome this, a fraction of the OTA present in the organic solvent is portioned to an aqueous buffer suitable for use with antibodies. This necessity results in losses in terms of the amount of OTA present in the sample matrix and the amount available for analysis.

Given the emerging regulatory requirements for OTA in the low ppb range, the manner in which a material is sampled is important for statistical relevance. Too small a sample will decrease the validity of extrapolation to a larger amount of potentially contaminated material. For this reason, commercial sampling is usually performed on relatively large amounts of material, up to a kg or more. This results in a large amount of solvent containing OTA at dilute concentrations. The current state-of-the-art involves a partitioning of the OTA from an organic solvent into an aqueous buffer and subsequent concentration of the OTA through the use of an immuno-affinity column. The OTA eluted from such a column is then analyzed through the use of high performance liquid chromatography (HPLC). It is also known in the art that the eluted OTA could be quantitatively analyzed through the use of specific antibodies through an ELISA approach. The sensitivity achieved through the combination of an immuno-affinity column followed by quantitative immuno detection is currently only in the 4 ppb range, and therefore not applicable to the lower level of emerging regulator requirements for OTA.

U.S. Pat. No. 5,475,096 teaches a method for the in vitro selection of DNA or RNA molecules that are capable of binding specifically to a target molecule. U.S. Pat. No. 5,631,146 teaches how to use this method to select a single stranded DNA molecule (oligonucletide) that is capable of binding specifically to adenosine molecules.

To our knowledge, no one to date has invented a DNA ligand that binds to any mycotoxin. In part this has been due to the inability of others to identify DNA ligands that bound to small molecule targets with sufficient affinity to enable sensitivity at a level required for commercial application.

The present invention relates to several novel strategies to improve the binding capacity of a DNA ligand to a small target molecule. Thus this invention enables the development of DNA ligands for mycotoxins with sufficient binding affinity to support detection of mycotoxins at levels that are relevant for food safety testing.

### SUMMARY OF THE INVENTION

The present invention provides an enablement of the identification and use of novel DNA ligands that bind mycotoxins for the determination of mycotoxin presence, concentration in samples, such as agricultural products, or for the removal of said mycotoxins from said sample. One embodiment of the invention would be the use of DNA ligands in an affinity column to partition the mycotoxins from the remainder of chemicals within agricultural products with the simultaneous concentration of such mycotoxins in solution, thus enabling quantitative determination. Certain mycotoxins such as ochratoxin A, and aflatoxin B1 exhibit sufficiently strong fluorescence to enable direct detection using fluorescence measurements following purification and concentration with an affinity column. For other mycotoxins, other methods of detection such as high performance liquid chromatography approaches would be required.

Thus in one aspect of the present invention a DNA ligand is provided wherein said DNA ligand binds to a mycotoxin. Mycotoxins include, without limitation, deoxynivalenol, zearalenone, T2-toxin, aflatoxin B1, fumosins, patulin and ochratoxin A. In one aspect of the present invention the mycotoxin is ochratoxin A.

According to one aspect of the present invention a DNA ligand that binds to ochratoxin A is provided. In one aspect the DNA ligand for ochratoxin A comprises at least one of the nucleotide sequence selected from the group consisting of the nucleic acid sequence set forth in the Sequence Listing as SEQ ID NOs: 1 to 12, SEQ ID NOs: 18 to 22 and functional analogues thereof. In another aspect of the present invention the DNA ligand that binds to ochratoxin A includes the nucleotide sequence set forth in the Sequence Listing as SEQ ID NO: 13 and functional analogues thereof.

According to still another aspect of the present invention a method for detecting a mycotoxin in a sample is provided, wherein the method comprises: (a) contacting said sample to a DNA ligand that binds to said mycotoxin under conditions wherein a mycotoxin/DNA ligand complex is formed if said mycotoxin is present in the sample; and (b) using detection means to determine whether said mycotoxin/DNA ligand complex is formed, thereby detecting the mycotoxin in the sample. In aspects of the present invention the method may be used for the detection, quantitation, removal or purification of the mycotoxin in or from the sample. In one aspect, the method for detecting a mycotoxin in a sample is characterized in that step (b) comprises partitioning said mycotoxin from the mycotoxin/DNA complex and using detection means to determine whether said mycotoxin is present in the sample.

According to a further aspect of the present invention, a method for determining the concentration of a mycotoxin in a sample is provided. Said method comprises the steps of: (a) contacting the sample to a DNA ligand that binds to said mycotoxin under conditions wherein a mycotoxin/DNA ligand complex is formed if said mycotoxin is present in the sample; and (b) measuring the amount of said DNA ligand by quantitatively detecting the mycotoxin/DNA ligand complex, thereby determining the concentration of the mycotoxin in the sample. In one aspect the method for determining the concentration of a mycotoxin in a sample is characterized in that step (b) comprises partitioning said mycotoxin from the mycotoxin/DNA complex and using detection means to measure the amount of said mycotoxin.

According to still another aspect of the present invention a method for removing or reducing the level of a mycotoxin present in a sample is provided wherein said method comprises contacting said sample to a DNA ligand that binds to said mycotoxin under conditions wherein a mycotoxin/DNA ligand complex is formed if said mycotoxin is according to still a further aspect of the present invention, a method for modifying the biological function of a mycotoxin is provided wherein said method comprises contacting said mycotoxin to a DNA ligand that binds to said mycotoxin under conditions wherein a mycotoxin/DNA ligand complex is formed.

According to yet another aspect of the present invention a method is provided to determine the concentration of a mycotoxin in a sample wherein said method comprises: (a) applying an extract of the sample to an affinity column having a DNA ligand which selectively binds mycotoxins thereby separating the mycotoxin from the sample; (b) detecting a signal from the mycotoxin separated from the sample; and (c) determining the concentration of mycotoxin present in the sample.

In one aspect, the extract of the sample is an organic solvent extract of the sample. Organic solvents that may be used in accordance with the present invention include, but are not limited to, methanol, ethanol and acetonitrile.

Fluorescence-based means may be used to detect the presence of the mycotoxin and for measuring the concentration of the mycotoxins in samples. Other detection means that can be used in aspects of the present invention include without limitation high performance liquid chromatography and mass spectrometry of the mycotoxin target, and the use of fluorescence, or fluorescence in combination with quenchers, or fluorescence polarization, and electro-affinity analysis of the target/DNA ligand complex formation. Samples include, without limitation, agriculture products, including crude grain extracts, and alcoholic beverages. In one aspect of the present invention, the concentration of ochratoxin A may be determined on the basis of a spectral shift in the fluorescence spectrum of ochratoxin A due to the binding of ochratoxin A to the DNA ligand.

According to a further aspect of the present invention, a method for the quantitative determination of a mycotoxin in a sample is provided, wherein the method comprises the steps of (a) reacting known concentrations of the mycotoxin with a DNA ligand that binds to said mycotoxin under conditions wherein a mycotoxin/DNA ligand complex is formed to construct a calibration curve representing the relationship between the fluorescence absorbance of the mycotoxin/DNA ligand complex and the concentration of the mycotoxin; (b) causing a sample having an unknown mycotoxin concentration to contact the DNA ligand that binds to said mycotoxin under conditions wherein a mycotoxin/DNA ligand complex is formed and measuring the fluorescence absorbance of the resulting reaction mixture; and (c) calculating the mycotoxin concentration in the sample by comparing the measured value obtained in step (b) with the calibration curve.

According to still another aspect of the present invention, uses of DNA ligand that bind to a mycotoxin in a method for determining the presence, concentration or the removal of the mycotoxin in or from a sample are provided.

According to a further aspect of the present invention, a composition comprising a cation for enhancing the affinity of a nucleic acid ligand to a target is provided, wherein said cation is selected from the group consisting of: sodium, potassium, calcium, scandium, titanium, vanadium, chromium, manganese, iron, cobalt, nickel, copper and zinc. In one aspect of the present invention, a composition comprising a calcium cation for enhancing the affinity of a nucleic acid ligand to a target is provided. In one aspect said target is ochratoxin A.

The DNA ligands of the present invention provide significant advantages over prior art methods for the concentration and detection of mycotoxins in sample material, including:
a. DNA ligands can be chemically synthesized. As the scale of production increases the relative cost per unit of DNA ligand is reduced.
b. DNA ligands can be modified directly through the covalent attachment of fluorophores or fluorescence quenching moieties. Due to their relatively large mass, antibodies quench fluorescence. This means that DNA ligands can be modified in order to directly measure the binding interaction between DNA ligand and ligand. Quantitative measurements with antibodies rely on indirect measurements such as competition analysis. This reduces sensitivity and increases cost.
c. Oligonucleotides can maintain function within higher levels of organic solvent than antibodies. This means in the case of target molecules where extraction must be performed with organic solvents, the use of DNA ligands allows more effective partitioning of the target molecule from the organic phase to a combined organic/aqueous buffer.
d. DNA ligands are more thermal stable than antibodies and can be stored for longer periods of time without a noticeable loss of function.

In general, it would be clear to one trained in the art that a DNA ligand that bound with high affinity and specificity to OTA would represent an improvement over existing antibody based methods both for the concentration of OTA prior to analysis, and for the direct, quantitative analysis of OTA concentration in sample material.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 is a graph illustrating the proportion of oligonucleotides eluted by buffer containing free OTA relative to total amount of oligonucleotides loaded onto column per selection cycle.

FIG. 2 is a graph illustrating the binding affinity of the DNA ligand OTA1.12 (SEQ ID NO: 1) to OTA with equilibrium dialysis over a range of DNA ligand concentrations.

FIG. 3 is a graph illustrating a comparison of OTA1.12 DNA ligand (SEQ ID NO: 1) affinity for OTA versus OTB with equilibrium dialysis. L=measurements from loading chamber of dialysis tube; R=measurements from the receiving chamber of dialysis tube.

FIG. 4 is a graph illustrating a comparison of crude grain extract and OTA1.12 (SEQ ID NO: 1) DNA ligand binding to OTA. L=measurements from loading chamber of dialysis tube; R=measurements from the receiving chamber of dialysis tube. OTA control refers to the loading of OTA alone in the loading chamber of the dialysis tube. OTA/grain refers to the loading of a combination of crude grain extract and OTA in the loading chamber of the dialysis tube. OTA/DNA ligand refers to the loading of a combination of OTA and DNA ligand in the loading chamber of the dialysis tube. Error bars represent a standard deviation from mean values.

FIG. 5 is a graph illustrating a comparison of OTA binding to OTA1.12 (SEQ ID NO: 1) DNA ligand in the presence and absence of crude grain extract.

FIG. 6 are graphs illustrating the effect of salts and buffer on the normalized fluorescence excitation (a) and emission (b) spectra of water solutions of 200 nM OTA in the presence of (1) 10 mM NaCl, (2) 10 mM KCl, (3) 5 mM HEPES pH 7.0, (4) 10 mM MgCl2. Spectra were normalized on the basis of the area under the curve.

FIG. 7 is a graph illustrating the fluorescence emission spectra of solutions of 200 nM OTA in the absence (1), or presence (2) of 5 μM EDTA, and in the presence (3) of 2 μM DNA ligand OTA1.12.6 (SEQ ID NO: 23). λexc=375 nm.

FIG. 8 are graphs illustrating the effect of pH on the excitation (a, b) and emission (c,d) spectra of 200 nM OTA solution containing 120 mM NaCl, 5 mM KCl and 5 mM MgCl2 in absence of DNA (a, c), with 2 μM DNA ligand OTA1.12 (b, d) (SEQ ID NO: 1). 1. pH=5.4 (10 mM acetate buffer); 2. pH=7.0 (10 mM HEPES) and 3. pH=8.5 (10 mM phosphate buffer). Excitation spectra were recorded in a microplate using λem=430 nm and bandpass for emission and excitation wavelengths of 20 and 5 nm respectively. Emission spectra were recorded using λexc=375 nm and bandpass for emission and excitation wavelengths of 5 and 20 nm respectively.

FIG. 9 is a graph illustrating the normalized fluorescence excitation spectra of OTA (1) and OTB (2). Spectra were normalized on the basis of the area under the emission curves.

FIG. 10 is a Stern-Volmer plot of the fluorescence quenching of OTA by potassium iodide (solid circles, Ksv=15.4±0.4 M-1) or OTA with 2 μM DNA ligands 1.12 (SEQ ID NO: 1, solid squares, Ksv=0.78±0.16 M-1), 1.12.5 (SEQ ID NO: 22, diamonds, Ksv=1.2±0.1 M-1), 1.12.6 (SEQ ID NO: 23, squares, Ksv=18.9±0.7 M-1) or OTA with 5 μM EDTA (triangles, Ksv=20.6±0.1 M-1). KCl was added to ensure the ionic strength was the same in all the solutions. Each point is the mean of two independent experiments. Stern-Volmer constant (Ksv) was calculated fitting the data to the equation Fo/F=1+Ksv[Kl], where F and Fo are the fluorescence of the solutions with or without the quencher respectively and [Kl] is the concentration of the quencher.

FIG. 11 is a graph illustrating the effect of varying level of organic solvents on the binding of OTA by the DNA ligand OTA1.12 (SEQ ID NO:1). Closed diamond=methanol, open square=ethanol, closed diamond=acetonitrile.

### DETAILED DESCRIPTION OF THE INVENTION

The details of certain embodiments of the invention are provided in the accompanying description herein. It is understood that one of ordinary skill in the art to which this invention belongs could envision other methods and materials similar or equivalent to those described herein can be used in the practice or testing of the present invention.

Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

The inventors have developed and identified novel nucleic acid ligands that specifically bind to a mycotoxin. It is understood by those skilled in the art that the novel nucleic acid ligands of the present invention may be involved in a variety of applications characterized by the binding of the nucleic acid ligands of the present invention to the mycotoxin.

DNA ligand means a DNA molecule that binds another molecule (target), such as a mycotoxin. In a population of candidate DNAs, a DNA ligand is one which binds with greater affinity than that of the bulk population.

In one embodiment of the invention, the inventors were able to identify novel DNA ligands that specifically bind to mycotoxins. In one aspect, the novel DNA ligands of this invention may be identified using PCR-based methods for identifying DNA ligands for a specific target. A mycotoxin target molecule, such as ochratoxin A is immobilized on a resin. A library of single stranded oligonucleotides each composed of a central region of random nucleotides flanked by sequences of known composition is applied to the immobilized mycotoxin in a column. Those oligonucleotides that do not bind to the immobilized mycotoxin, or bind relatively weakly are removed through repeated washes of the column with a buffer that supports DNA ligand binding. Those oligonucleotides that do bind with high affinity to the immobilized mycotoxin are recovered through the addition of an excess of free molecules of the same mycotoxin being selected for. This elution process also provides a selection pressure for DNA ligand specificity. The recovered putative DNA ligands are PCR amplified, the sense strand is purified from the antisense, and re-applied to a fresh column containing the immobilized target mycotoxin, where the process described above is repeated. This process is repeated until no further selection gains are evident in the population of oligonucleotides at which point the library is amplified, cloned and individual oligonucleotides are sequenced. Putative DNA ligands are synthesized based on the sequences discovered and tested for their ability to bind to the free mycotoxin target.

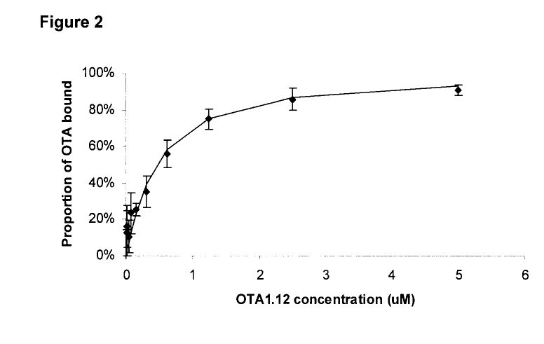

Thus in one embodiment the present invention relates to DNA ligands that specifically bind to mycotoxins. In one aspect of the present invention, the inventors were able to identify DNA ligands that specifically bind the mycotoxin ochratoxin A (OTA). Using binding assays, the inventors demonstrated that the DNA ligands selected for binding to OTA bound with more affinity to OTA than DNA ligands that were selected for other non-mycotoxin targets, such as a DNA ligand selected for sulforhodamide (SEQ ID NO: 16). These other DNA ligands would be considered by one trained in the art as essentially random, as they were not selected following exposure to OTA. While the random DNA ligand for sulforhoamide had a low affinity for OTA, OTA bound the DNA ligands of the present invention with a strikingly high affinity (Table 1 and FIG. 2).

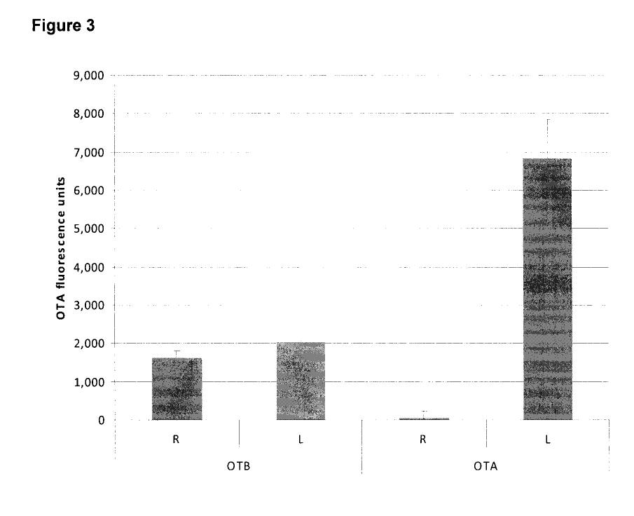

The inventors' results demonstrate that the DNA ligands of the present invention are specific for the mycotoxin that they were selected for. Using an equilibrium dialysis experiment, the inventors showed that DNA ligands selected for OTA have close to 700 fold greater affinity for OTA than for ochratoxin B (FIG. 3).

The DNA ligands of the present invention may also encompass “functionally equivalent variants” or “analogues” of the oligonucleotides. As such, this would include but not be limited to oligonucleotides with partial sequence homology, oligonucleotides having one or more specific conservative and/or non-conservative base changes which do not alter the biological or structural properties of the DNA ligand (i.e. the ability to bind to a mycotoxin).

In terms of “functional analogues”, it is well understood by those skilled in the art, that inherent in the definition of a biologically functional oligonucleotide analogue is the concept that there is a limit to the number of changes that may be made within a defined portion of the molecule and still result in a molecule with an acceptable level of equivalent biological activity, which, in this case, would include the ability to bind to a mycotoxin. A plurality of distinct oligonucleotides with different substitutions may easily be made and used in accordance with the invention. It is also understood that certain bases are particularly important to the biological or structural properties of the DNA ligand in the mycotoxin recognition region, such bases of which may not generally be exchanged.

The DNA ligand analogues of the instant invention also encompass oligonucleotides that have been modified by the inclusion of non-natural nucleotides including but not limited to, 2,6-Diaminopurine-2′-deoxyriboside, 2-Aminopurine-2′-deoxyriboside, 6-Thio-2′-deoxyguanosine, 7-Deaza-2′-deoxyadenosine, 7-Deaza-2′-deoxyguanosine, 7-Deaza-8-aza-2′-deoxyadenosine, 8-Amino-2′-deoxyadenosine, 8-Amino-2′-deoxyguanosine, 8-Bromo-2′-deoxyadenosine, 8-Bromo-2′-deoxyguanosine, 8-Oxo-2′-deoxyadenosine, 8-Oxo-2′-deoxyguanosine, Etheno-2′-deoxyadenosine, N6-Methyl-2′-deoxyadenosine, O6-Methyl-2′-deoxyguanosine, O6-Phenyl-2′-deoxyinosine, 2′-Deoxypseudouridine, 2′-Deoxyuridine, 2,4-Difluorotoluyl, 2-Thiothymidine, 4-Thio-2′-deoxyuridine, 4-Thiothymidine, 5′-Aminothymidine, 5′-Iodothymidine, 5′-O-Methylthymidine, 5,6-Dihydro-2′-deoxyuridine, 5,6-Dihydrothymidine, 5-(C2-EDTA)-2′-deoxyuridine, 5-(Carboxy)vinyl-2′-deoxyuridine, 5-Bromo-2′-deoxycytidine, 5-Bromo-2′-deoxyuridine, 5-Fluoro-2′-deoxyuridine, 5-Hydroxy-2′-deoxycytidine, 5-Hydroxy-2′-deoxyuridine, 5-Hydroxymethyl-2′-deoxyuridine, 5-Iodo-2′-deoxycytidine, 5-Iodo-2′-deoxyuridine, 5-Methyl-2′-deoxycytidine, 5-Propynyl-2′-deoxycytidine, 5-Propynyl-2′-deoxyuridine, 6-O-(TMP)-5-F-2′-deoxyuridine, C4-(1,2,4-Triazol-1-yl)-2′-deoxyuridine, N4-Ethyl-2′-deoxycytidine, O4-Methylthymidine, Pyrrolo-2′-deoxycytidine, and Thymidine Glycol.

The DNA ligands of the present invention may be made by any of the methods known to those of skill in the art most notably, preferably by chemical synthesis. A common method of synthesis involves the use of phosphoramidite monomers and the use of tetrazole catalysis (McBride and Caruthers, Tetrahedron Lett. (1983) 24:245-248). Synthesis of an oligonucleotide starts with the 3′ nucleotide and proceeds through the steps of deprotection, coupling, capping, and stabilization, repeated for each nucleotide added.

Thus, according to the present invention DNA ligands obtained and characterized following the mycotoxin selection strategy outlined herein exhibit significant binding affinity for mycotoxins. In one aspect, DNA ligands obtained and characterized following the OTA selection strategy outlined herein exhibit significant binding affinity for OTA.

As previously shown in Table 1 all oligonucleotides obtained and characterized following the OTA selection strategy outlined herein exhibited significant binding affinity for OTA.

Table 2 demonstrates how the binding affinities for the various DNA ligands identified herein relate to the presence of a consensus motif.

For the most part the closer the sequence approaches the consensus motif the greater the binding affinity for OTA (the lower the kD). Sequence variation within the DNA ligands identified in this present invention is constrained to the about 40 nucleotide interior random sequence in the original library. This sequence consensus is most evident within a consensus motif consisting of the nucleotide sequence GATCGGGTGTGGGT (SEQ ID NO: 13). It would be clear to anyone trained in the art of the present invention that the emergence of a consensus motif of 12 to 14 nucleotides within a random region of about 30 to about 45 nucleotides is meaningful in terms of selection for binding to OTA.

To develop improved DNA ligands for OTA, the inventors designed and synthesized a series of four shorter oligonucleotides based on sequence OTA1.12 (SEQ ID NO: 1) (Sigma Genosys). These are listed as SEQ ID NOs: 17 to 23. All seven oligonucleotides (SEQ ID NOs: 17 to 23) as well as an oligonucleotide of SEQ ID NO: 1 were tested for OTA binding capacity with equilibrium dialysis under conditions identical to those detailed in the Examples provided herein. Table 3 provides the estimate of the binding affinity (kD) that was obtained based on this analysis for each oligonucleotide.

Clearly the shortened DNA ligand, OTA1.12.2 (SEQ ID NO: 19) provided the optimum balance between a reduction in oligonucleotide size (a cost saving for synthesis of the molecule) while maintaining binding activity.

Thus, according to the present invention truncated oligonucleotides designed from DNA ligands obtained and characterized following the OTA selection strategy outlined herein exhibit significant binding affinity for OTA. In one aspect of the present invention, the truncated DNA ligands for OTA comprise the following sequences:

(SEQ ID NO: 18)
GATCGGGTGT GGGTGGCGTA AAGGGAGCAT CGGACAACGA T
(SEQ ID NO: 19)
GATCGGGTGT GGGTGGCGTA AAGGGAGCAT CGGACA
(SEQ ID NO: 20)
GATCGGGTGT CGGTGGCGTA AAGGGAGCAT CGGACAACGA T
(SEQ ID NO: 21)
GATCGTGTGT CGGTGGCGTA AAGGGAGCAT CGGACAACGA T
(SEQ ID NO: 22)
GATCGGGTGT GGGTGGCGTA AAGGGAGCAT CGGACAACG

In another embodiment of the present invention, the inventors discovered that the removal of magnesium from the mycotoxin/DNA ligand binding buffer (10 mM HEPES pH 7.0; 120 mM NaCl, 5 mM KCl, 5 mM MgCl₂) with the addition of calcium enhances binding affinity. Other researchers have predominantly relied on the use of magnesium for the stabilization of the DNA structure. It has been previously shown (Sazani et. al., J. Am. Chem. Soc., (2004) 126, 8370-8371) that magnesium was a necessary element for DNA ligand/ATP binding through the formation of a bridge between the two molecules. The present inventors have been the first to realize and demonstrate that calcium is capable of forming a better molecular bridge between ochratoxin A and a DNA ligand than magnesium is. This important discovery resulted in a decrease in the coefficient of disassociation (Kd) from approximately 150 nM with 10 mM Mg to approximately 50 nM with 10 mM calcium.

However, the affinity between OTA and the DNA ligand was not affected significantly by the concentration of sodium or potassium (data not shown), to the point that complete removal of sodium or potassium from the binding buffer did not affect the affinity as long as magnesium or calcium were present. This indicates the ionic strength of the solution had little effect on the interaction between OTA and the DNA ligand.

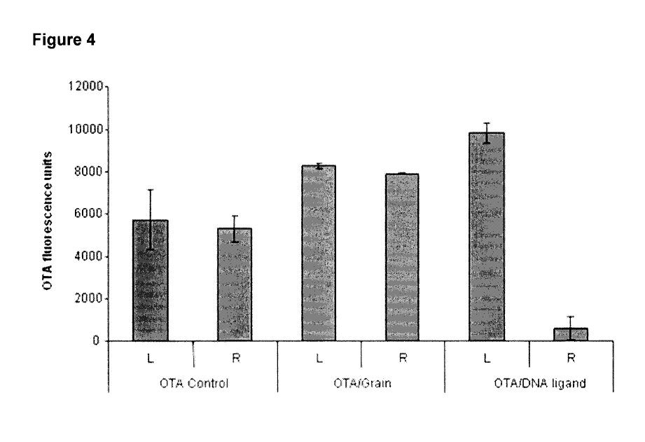

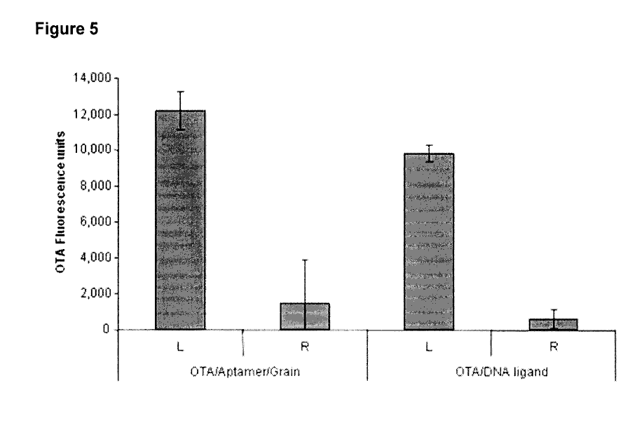

One potential constraint to the use of the present invention as a rapid diagnostic tool for the quantitative determination of mycotoxin concentration in sample extracts such as grain extracts is the potential for other compounds from a crude extract interfering with the binding reaction. This could occur as a result of compounds from the crude grain extract binding to the DNA ligand. This could also occur as a result of compounds from the crude grain extract binding to the mycotoxin and interfering or altering the ability of the DNA ligand to bind to the mycotoxin. The inventors successfully eliminated this concern by demonstrating that a crude grain extract does not affect OTA binding to the DNA ligand, and that compounds extracted from grain do not bind significantly to the DNA ligand of the present invention (FIG. 4 and FIG. 5).

The novel nucleic acid ligands for mycotoxins of the present invention may be involved in a variety of applications characterized by the binding of the nucleic acid ligands of the present invention to mycotoxins.

Determining the Presence and/or Concentration of Mycotoxins in Samples

In one aspect the DNA ligands of the present invention are used for the quantitative determination of mycotoxin concentration in samples of interest, including but not limited to, deoxynivalenol, zearalenone, T2-toxin, aflatoxin B1, fumosins, patulin and ochratoxin A. In another aspect, the DNA ligans of the present invention may be used to determine the presence or absence of a mycotoxin in a sample. In yet another aspect, the DNA ligands of the present invention may be used to remove or reduce the level of mycotoxins in a sample.

It would be clear to one trained in the art that several methods exist that would enable the potential use of the DNA ligands for a mycotoxin of the present invention for the determination of the concentration of that mycotoxin in a sample. In one aspect the present invention includes methods for detecting the presence of a mycotoxin in a sample, said method comprising: contacting said sample to a DNA ligand that binds to said mycotoxin under conditions wherein a mycotoxin/DNA ligand complex is formed if said mycotoxin is present in the sample; and using detection means to determine whether said mycotoxin/DNA ligand complex is formed, thereby detecting the mycotoxin in the sample. In another aspect the present invention includes methods for determining the concentration of a mycotoxin in a sample, said method comprising: contacting said sample to a DNA ligand that binds to said mycotoxin under conditions wherein a mycotoxin/DNA ligand complex is formed if said mycotoxin is present in the sample; and measuring the concentration of said mycotoxin/DNA ligand complex, thereby determining the concentration of the mycotoxin in the sample.

For example, the inventors have demonstrated the efficacy of the use of a DNA ligand for OTA, for determining the concentration of the OTA in an agricultural product through the use of an affinity column. As such, one embodiment of this invention provides for the use of DNA to determine the concentration of OTA.

The immobilization of DNA ligands and their subsequent use for determination of concentration of the target molecule that the DNA ligand binds to in a sample has been achieved prior to this invention. Romig et al. (J. Chromatogr. (1999) B 731:275-284) immobilized a 5′ biotinylated DNA ligand for human L-selectin onto a streptavidin sepharose support which was then packed into a column. The target protein was eluted from the column under conditions that did not cause protein denaturation, but affected the cation support of the tertiary structure necessary for DNA ligand binding. This application resulted in a 1,500 fold purification of the target protein, with 83% recovery in a single step. Kotia et al. (Anal Chem (2000) 72:827-831) has demonstrated that immobilized DNA ligands can be used to concentrate small target molecules similar in size to OTA. This group demonstrated that immobilized DNA ligands could be used to separate polyaromatic hydrocarbons such as naphthalene and benzo[a]pyrene (BaP), as well as naphthalene and benzo(ghi)perylene (BgP). Kotia et al. demonstrated that separation results were improved through the use of acetonitrile concentrations up to 60% for BaP, and methanol concentrations from 20 to 30% for BgP. The inventors of the present invention realized that the development of similar technology for the detection of mycotoxins would be useful given that the extraction of mycotoxins relies on the use of organic solvents such as methanol, ethanol or acetonitrile.

For use with immunoaffinity columns the OTA must be partitioned from these organic solvents into an aqueous solvent. This step requires additional time, and results in both an increased dilution of the target molecule and implicit losses of OTA from the analysis procedure.

An inventive step in the application of this platform to the determination of mycotoxin concentration has been the demonstration by the inventors of the use of relatively high levels of organic solvents in the affinity column without a compensatory loss in DNA ligand binding activity with a mycotoxin target. This inventive step provides a significant advantage over antibody based methods which require substantially more dilution of the organic solvents used in extraction prior to exposure to the antibody.

As such, one embodiment of this invention includes the use of a DNA ligand in an affinity column for the determination of mycotoxin presence and/or concentration in a sample comprising the following steps:
(a) immobilizing a DNA ligand for a mycotoxin to an affinity column;
(b) running an extract of the sample through the affinity column under conditions wherein a mycotoxin/DNA ligand complex is formed if said mycotoxin is present in the sample;
(c) recovering the mycotoxin from the column with a recovering agent; and
(d) measuring the quantity of mycotoxin captured by the column by methods such as direct fluorescence measurement, high performance liquid chromatography and mass spectrometry of the mycotoxin target, and the use of fluorescence, or fluorescence in combination with quenchers, or fluorescence polarization, and electro-affinity analysis of the target/DNA ligand complex formation.

In one aspect of the invention the extract is an organic solvent extract of the sample. Suitable organic solvents include, but are not limited to, methanol and ethanol. In another aspect the organic extract solution may be diluted to a level where the organic solvent is tolerated by the DNA ligand (for example, 5% to 25% methanol, or 10% ethanol). The recovering agent may comprise 20% methanol without salts or 10% ethanol.

In another embodiment of this invention the method for the determination of mycotoxin concentration would include a washing step following the introduction of the sample to the affinity column and prior to the elution of the sample from the column.

Another embodiment of this invention would include a sample pretreatment step to reduce the concentration of potential contaminating compounds.

Another embodiment of this invention comprises the use the DNA ligands for ochratoxin A in alcoholic beverages such as wine. The stability of the DNA ligand/mycotoxin binding in 10% ethanol allows for direct analysis of alcoholic beverages such as wine following an appropriate pre-cleaning step for removal of contaminating phenolic compounds.

Given that the DNA ligands of the present invention when in contact with a sample bind only to mycotoxins that may be present in the sample to form a mycotoxin/DNA ligand complex, another aspect of the present invention comprises methods for removing or reducing the level of mycotoxins in the sample. Furthermore, given that the DNA ligands of the present invention when in contact with a sample bind only to mycotoxins that may be present in the sample to form a mycotoxin/DNA ligand complex, the DNA ligands of the present invention may be used in a method for modifying the biological function of the mycotoxin, including the inhibition of the biological function of the mycotoxin. Therefore another aspect the present invention comprises methods for modifying the biological function of mycotoxins.

Given that a DNA ligand based affinity column would bind only the mycotoxin present within a sample while allowing other components of an agricultural product to flow through, an embodiment of the present invention would be the use of affinity columns consisting of DNA ligands for mycotoxins for the removal, or reduction of mycotoxins in agricultural products. One embodiment of this invention would be the removal or reduction of ochratoxin A in agricultural products through the use of an affinity column.

Fluorescence-based means may be used to detect the presence of the mycotoxin and for measuring the concentration of the mycotoxins in samples. Other detection means that can be used in aspects of the present invention include without limitation high performance liquid chromatography and mass spectrometry of the mycotoxin target, and the use of fluorescence, or fluorescence in combination with quenchers, or fluorescence polarization, and electro-affinity analysis of the target/DNA ligand complex formation. In one aspect of the present invention, the concentration of ochratoxin A may be determined on the basis of a spectral shift in the fluorescence spectrum of ochratoxin A due to the binding of ochratoxin A to the DNA ligand.

Samples include materials that may contain mycotoxins, including, without limitation, agriculture products, including crude grain extracts, and alcoholic beverages.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Identification of DNA Ligands for a Mycotoxin

In the present invention an initial library was created with two regions of known sequence flanking 30 nucleotides of unknown sequence. The two regions of known sequence were used as complementary sites for PCR amplification with the primers listed as SEQ ID NO: 14 and SEQ ID NO: 15. A quantity of this library was used that would correspond to 10¹⁵ sequences for the initial round of selection. OTA (Romer Labs™) was dissolved to a concentration of 5 μmol in 200 μL of DMSO (10 mM) and mixed with 500 μmol of 1-Ethyl-3-[3-dimethylaminopropyl]carbodiimide hydrochloride (EDC, Sigma E1769) in 1 mL of water. Immediately after mixing these compounds, 5 mL of DADPA (Pierce Biotechnology, Pierce No. 20266) slurry in 20 mM phosphate buffer pH 5.0 was added and the entire mixture was then rotated for 1 h at room temperature. To quench un-reacted amino groups, the resin was equilibrated with carbonate buffer pH 8.5 and 300 μmol of sulfo NHS-acetate (Pierce Biotechnology, Pierce No. 26777) was added to the slurry. The mixture was rotated for another hour after which the resin was washed extensively with OTA Selection Buffer (10 mM HEPES pH 7.0; 120 mM NaCl, 5 mM KCl, 5 mM MgCl₂). The presence of OTA in the resin was corroborated by the intense fluorescence of the resin when irradiated with UV light at a wavelength of 366 nm.

Simultaneously, a resin for negative selection was prepared by adding 75 μmol of sulfo NHS-acetate to 1 mL of resin and subsequently equilibrated in carbonate buffer pH 8.5. The library was denatured by heating at 90° C. for 5 min, and renatured for 30 min at room temperature prior to column loading. Both positive (OTA-immobilized resin) and negative columns were prepared by loading 250 μL of the resin in slurry into a disposable microspin column (Bio-Rad). This resulted in a column volume (CV) of approximately 200 μL. The resin was washed extensively with Selection Buffer and the library was loaded in the column and incubated for varying time periods. The column was then washed with 12 CV of selection buffer. Oligonucleotides that bound to the OTA-immobilized resin were eluted with three sequential incubations of 10 min each with a 100 μL solution comprised of 2 mM OTA in Selection Buffer containing 2.5% of DMSO. Each of the three elution fractions was maintained separately for PCR amplification. Each fraction was concentrated through the use of 10 KDa microcon filters (Millipore). The eluted fraction was PCR amplified with SEQ ID NO: 14 (with a 5′ Cy3 fluorophore), and SEQ ID NO: 15 (with a 5′ biotin) in a solution comprised of five units of Taq DNA polymerase, 10 mM Tris/HCl (pH 8.3), 50 mM KCl, 1 mM MgCl2, 200 μM of each dNTP (GTP, ATP, CTP and TTP). Each amplification reaction was driven by 1 μM of each primer. Amplifications were performed under the following conditions; an initial denaturation step of 5 min at 94° C., followed by a varying number of cycles consisting of denaturation at 94° C. for 10 sec., annealing at 54° C. for 15 sec. and extension at 72° C. for 20 sec. These cycles were followed by a final extension phase of 3 min at 72° C. after which samples were held at 40° C. until they were processed. Following PCR amplification an aliquot from each tube was removed and the sample was analyzed by polyacrylamide electrophoresis on a 10% acrylamide vertical gel (Bio-Rad). Gels were stained with ethidium bromide, and the presence of amplified fragments of the appropriate size was evaluated through comparison to a molecular weight ladder.

Streptavidin-agarose resin was used in combination with the biotin label present on the anti-sense primer to separate single stranded, sense copies of the putative DNA ligands. Sodium chloride was added to the PCR products to a concentration of 1 M. The streptavidin-agarose resin was activated by washing with a PBS buffer three times at room temperature. The activated resin was then combined with the double stranded PCR products, and incubated for 30 min at room temperature in the dark in the rotator, followed by three washes with the PBS buffer, and one wash with the Selection Buffer. Following these washes, 50 μl of Selection Buffer was added to the resin, and the sense strands were liberated through denaturation at 95° C. for 10 min. The slurry was then microcentrifuged and the supernatant removed to a fresh tube. This denaturation step was repeated once, and the recovered sense strand fragments were combined.

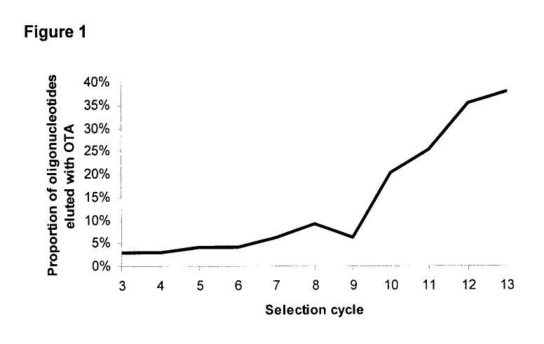

Starting with the third cycle of selection, counter selection for targets similar to OTA was incorporated into the selection strategy. Following the 12 CV of washes with Selection Buffer the following counter selection agents were introduced sequentially, a crude grain extract, warfarin (2 mM) and N-acetylphenyl alanine (5 mM). The crude grain extract was prepared by adding 1 g of ground wheat grain to 10 mL of Selection Buffer and rotating the extraction solution for 1 h. The crude grain extraction solution was centrifuged and the supernatant decanted through a 0.45 μm filter. Following filtration the crude grain extraction solution was diluted tenfold with Selection Buffer. Solutions containing the counter selection agents were incubated sequentially in the column for 10 min each. Following selection cycle 5 two sequential counter selection steps with the crude grain extract were incorporated into the selection strategy. After counter selection incubations were completed the column was washed with 6 CV of Selection Buffer. Oligonucleotides still retained by the OTA-immobilized resin were eluted in an identical manner to the first three selection cycles. Following the second cycle of selection the first two eluates were combined and used to estimate the proportion of DNA ligands eluted with OTA. 5 μL of each collected fraction was mixed with 100 μL of water in a 96-well black microplate and the fluorescence (Cy3) was measured using λexc=540 nm and λem=580 nm. The third eluate was also combined with the first two eluates before all remaining eluate material was concentrated and washed with 10 KDa microcon filter (Millipore). The adjustment of variables within each selection cycle is described in FIG. 1. In the last selection cycle PCR was performed using unlabeled versions of Sequence ID#15 and 16. The PCR product was then ligated into pGEM-T vectors and cloned into E. coli to facilitate clone sequencing. Table 4 provides a summary of the number of PCR cycles performed per selection cycle, library incubation time with the OTA-immobilized resin, and identification of which cycles incorporated a negative selection and/or counter selection strategies.

### Binding Assays

A total of 12 putative DNA ligands were synthesized (Sigma Genosys) and screened for binding to OTA by equilibrium dialysis. Microequilibrium dialyzers (Harvard Apparatus) were loaded with selection buffer in the receiving chamber and selection buffer containing 200 nM OTA and 20 μM of a specific DNA ligand in the loading chamber. Dialysis was allowed to proceed for 48 h at room temperature. Two replicates were performed for each of the DNA ligands tested. The affinity of DNA ligands to OTA was estimated by measuring the intrinsic fluorescence of OTA in the loading (FI) and receiving (Fr) chambers. The fraction of bound OTA (f) was then determined as:

$$f = \frac{{F}_{l} - {F}_{r}}{{F}_{l}} \qquad ( 1 )$$

The dissociation constant (kD) was estimated as follows;

$$Kd = \frac{[ {A}_{0} ]}{f} - [ {A}_{0} ] \qquad ( 2 )$$

where [A₀] is the total concentration of the DNA ligand. Certain oligonucleotides that had been demonstrated either by the inventors or by others to bind to other targets were also tested with equilibrium dialysis for their ability to bind to OTA (SEQ ID NO: 16, an DNA ligand that binds to sulforhodamine (Wilson and Szostak (1998) Chem Biol. 5:609-617). It has been suggested (Ardus et al. (1998) Can. J. Chem. 76(6): 907-918) that OTA binds to supercoiled DNA and is capable of nicking a strand and causing linearization.

Table 1 shows that all of the oligonucleotides selected using the OTA selection protocol of the present invention bound OTA with greater affinity than to a DNA ligand selected for sulforhodamide. This other oligonucleotide would be considered by one trained in the art of the present invention as essentially random, as it was not selected following exposure to OTA. Clearly the sequences identified in this invention represent a qualitatively different level of binding between DNA and OTA than any interaction that may occur between OTA and random sequence DNA.

### Example 2

### More Precise Determination of kD for DNA Ligands for Mycotoxins

A more precise determination of the kD for of the DNA ligand described in the present invention was performed by equilibrium dialysis over a range of concentrations. A series of dialyzers were prepared with 200 nM OTA and a varying concentration of DNA ligand in the loading chamber (FIG. 2). The stoichiometry between OTA and the DNA ligands was assumed to be 1:1. The kD's were determined by fitting the experimental data to the equation 3 using the Levenberg-Marquardt algorithm and SigmaPlot 2001 program version 7.0 (SPSS Inc., Chicago, Ill.).

$$f = \frac{\begin{matrix}( [ {A}_{0} ] + [ {OTA}_{0} ] + Kd ) - \\ \sqrt{{( [ {A}_{0} ] + [ {OTA}_{0} ] + Kd )}^{2} - 4 [ {A}_{0} ] [ {OTA}_{0} ]}\end{matrix}}{2 [ {OTA}_{0} ]} \qquad ( 3 )$$

where [OTA0] is the starting or total concentration of OTA.

In this instance the kD of the DNA ligand OTA1.12 (SEQ ID NO: 1) was determined to be 360 nM±60 nM. This is in the range of binding affinities discovered by others for small molecule targets.

### Example 3

### Demonstration of Specificity of DNA Ligands in Present Invention for OTA

An equilibrium dialysis experiment was performed with OTA and OTB in equal concentrations (1 μM) with equal amounts (10 uM) of DNA ligand, OTA1.12 (SEQ ID NO: 1). This dialysis experiment was carried out under exactly the same conditions as the dialysis experiments described in Example 1. FIG. 3 shows that the DNA ligand OTA1.12 (SEQ ID NO: 1) has 692 fold greater affinity for OTA than OTB.

### Example 4

### Demonstration of Lack of Interference of DNA Ligand Interaction with OTA from Crude Grain Extract Material

A crude grain extract was prepared as described in the detailed description of the invention. FIG. 4 shows that the crude grain extract did not bind a significant amount of OTA. The difference between the amount of OTA fluorescence in the loading and receiving chambers was 8.0%±10% for the TA control experiments versus 4.5% ±1.3% for the grain extract. This does not represent a statistically significant difference.

This experiment showed that the grain extract is not measurably binding OTA. The amount of OTA fluorescence in the presence of DNA ligand differed between the two chambers by 93.8%, a result that was highly significant statistically (t test, P<0.01 (one way)).

The potential for the crude grain extract to interfere with OTA binding with the DNA ligand of the present invention was also tested. Equilibrium dialysis experiments were performed in the presence and absence of crude grain extract with identical amounts of OTA (0.4 μM) and DNA ligand (10 μM). Grain extract was added as a 10% v/v blend to the Selection Buffer. FIG. 5 provides a comparison of the OTA fluorescence measured in the various treatments arising from this experiment. It is clear that the grain extract does not measurably affect OTA binding to the DNA ligand of the present invention.

### Example 5

### Demonstration of OTA Binding to DNA Ligand of the Present Invention on the Basis of Modulation of OTA Fluorescence

OTA fluoresces over a broad peak with greatest intensity at 450 nm when excited with light at a wavelength of 379 nm. (Verrone et al., (2007) J Incl Phenom Macrocycl Chem 57:475-479). The interaction of OTA with the oligonucleotides developed in the present invention was investigated.

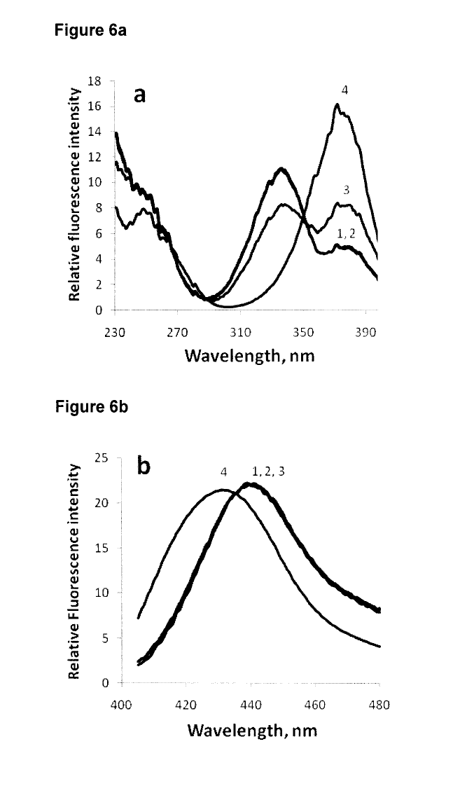

The shape of the absorption and fluorescent spectra of OTA in aqueous solutions at different pHs is determined by the ionization state of the molecule and has been described before (Illichev 2001; 2002). OTA has two pKa values: 4.2 and 7.2 (Chu 1971). At a pH close to neutral both the monoanion and dianion forms coexist while at alkaline pH both the carboxyl and the phenolic hydroxyl groups are ionized. This is reflected in the absorption and excitation spectra of the molecule that present peaks at 330 and 375 nm, at neutral pH; while at alkaline pH there is only an excitation peak at 375 nm. We found that adding 5 mM Mg²⁺ to an aqueous neutral solution of OTA dramatically affected the absorption and excitation spectra. The excitation peak at 330 nm disappeared while the fluorescence intensity of the peak at 375 nm increased twenty fold (FIG. 6a). This indicates that Mg²⁺ formed a complex with OTA and moved the protolitic equilibrium to the deprotonated form. Addition of Mg²⁺ also produced a blue shift of the emission spectrum (FIG. 6b). Other cations like Na⁺ or K⁺ increased only slightly the fluorescence intensity of the molecule and did not generate a wavelength shift in the emission spectrum.

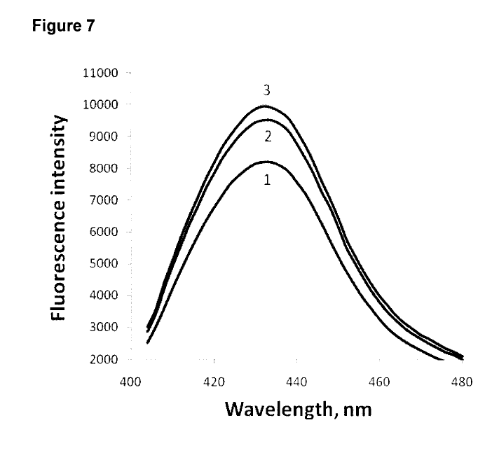

The fluorescence spectrum of OTA was affected dramatically by the presence of any DNA molecule. In the presence of DNA molecules that did not exhibit binding to OTA, the fluorescence intensity of the OTA molecule increased without a spectral shift (FIG. 7). We have shown that the addition of ethylenediaminetetraacetic acid (EDTA) produced a similar enhancement of OTA fluorescence as the addition of non-binding DNA. We determined whether the EDTA present in our DNA preparations was responsible for the observed change in fluorescence intensity of OTA in the presence of non-binding DNA. We washed specific non-binding DNA oligonucleotides five times in a 10 KDa microcon filter with distilled water. This DNA solution increased the fluorescence of OTA to a similar level as EDTA thus demonstrating that the DNA molecule itself exerted an effect on OTA fluorescence in the absence of binding.

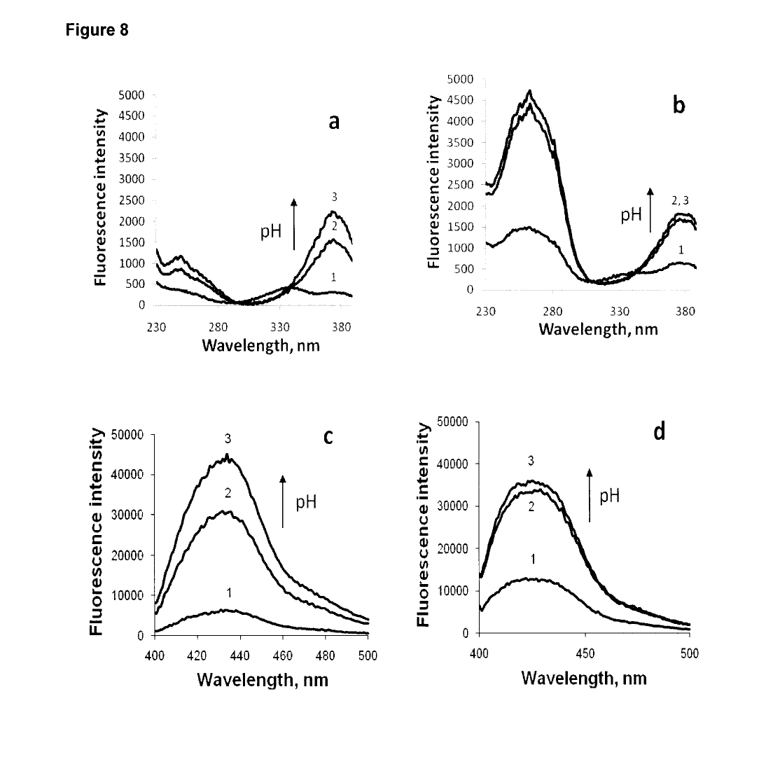

In the presence of DNA ligands that were shown to bind to OTA such as OTA1.12 (SEQ ID NO: 1) there was a 12 nm blue shift of the maximum emission peak indicating OTA was localized in a more hydrophobic environment (FIGS. 8.c and d). We plotted the spectral shift against the concentration of the DNA ligand 1.12 (SEQ ID NO: 1) and obtained a curve that could be fitted to the binding equation 3, replacing f by A/Amax, where A is the shift of the peak at a given DNA ligand concentration and Amax is the maximum shift. The Kd obtained with this curve coincided with that obtained by equilibrium dialysis.

The relationship found by the inventors between the spectral shift described before and the concentration of OTA may be used in a method for the quantitative determination of a mycotoxin in a sample wherein the method comprises the steps of (a) reacting known concentrations of the mycotoxin with a DNA ligand that binds to said mycotoxin under conditions wherein a mycotoxin/DNA ligand complex is formed to construct a calibration curve representing the relationship between the fluorescence absorbance of the mycotoxin/DNA ligand complex and the concentration of the mycotoxin; (b) causing a sample having an unknown mycotoxin concentration to contact the DNA ligand that binds to said mycotoxin under conditions wherein a mycotoxin/DNA ligand complex is formed and measuring the fluorescence absorbance of the resulting reaction mixture; and (c) calculating the mycotoxin concentration in the sample by comparing the measured value obtained in step (b) with the calibration curve.

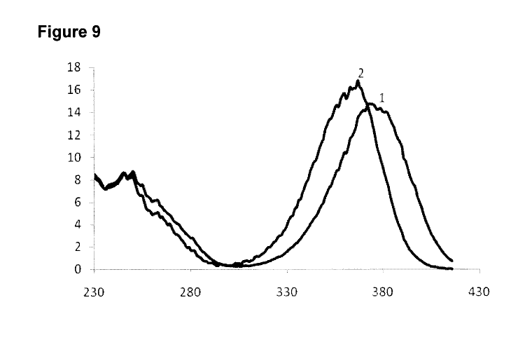

Another remarkable effect of the OTA/DNA ligand complex formation on OTA fluorescence was a dramatic increase of fluorescence at excitation wavelengths between 230 and 280 nm when the emission was set at 430 nm (FIGS. 8.a and b). This effect wasn't observed when either OTA or OTB were free in solution at any pH or salt concentration, suggesting it was a direct consequence of the configuration of the complex OTA/DNA ligand. As can be seen if compare the excitation spectra of OTA and OTB (FIG. 9) the UV band between 230 and 280 nm is very similar between the two molecules. OTA differs from OTB only in a chlorine atom attached to the phenol ring in the isocoumarin fragment of OTA. Chlorine affects the electronic structure of the molecule with a strong negative inductive effect and a positive mesomeric effect. These electronic effects shift the excitation peak of the isocoumarin from 365 nm in OTB to 375 nm in OTA but do not affect the UV excitation band located under 280 nm, indicating this band is mainly result of energy transfer from the phenylalanine moiety, which is identical between OTA and OTB. However, even though OTB binds to DNA ligands identified in this invention with low affinity (Kd=30 μM) the related UV band is not affected by binding. This suggests that the high affinity of OTA to DNA ligands is strongly regulated by the specific electronic and steric features of OTA and differs from the mechanism of OTB interaction with DNA.

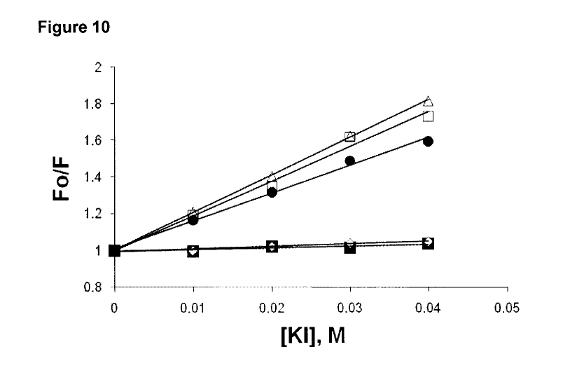

To further support that OTA was bound in a hydrophobic pocket we conducted quenching experiments with the hydrophilic collisional quencher potassium iodide. Fluorescence quenching of OTA in the presence of DNA ligands OTA1.12 or OTA1.12.5 (SEQ ID NO: 1 and SEQ ID NO: 22) was very low (FIG. 10) indicating the toxin was shielded in a hydrophobic pocket. Conversely, potassium iodide quenched OTA fluorescence when it was free in solution or in the presence of DNA ligands lacking affinity to OTA like 1.12.6 (SEQ ID NO: 23). Surprisingly, the Stern-Volmer constant was higher in the presence of non binding DNA or EDTA in the solution.

This demonstrates that the DNA ligands discovered in this invention for OTA can be used to determine OTA concentration on the basis of a spectral shift in OTA.

### Example 6

### Characterization of Sequence Dependency for Binding to OTA

To develop improved DNA ligands for OTA a series of shorter oligonucleotides based on the OTA1.12 sequence (SEQ ID NO: 1) were designed and synthesized (Sigma Genosys). These are listed as SEQ ID NOs: 17 to 23. All seven oligonucleotides (SEQ ID NOs: 17 to 23) as well as an oligonucleotide of SEQ ID NO: 1 were tested for OTA binding capacity with equilibrium dialysis under conditions identical to those detailed in the above Examples. Table 3 provides the estimate of the binding affinity (kD) that was obtained based on this analysis for each oligonucleotide.

### Example 7

### Use of a DNA Ligand Based Affinity Column to Purify, Concentrate and Determine the Concentration of OTA in Samples

The DNA ligand OTA1.12.2 (SEQ ID NO: 19) was conjugated through the 5′ phosphate group to the resin diaminodiprolpylamine agarose (DADPA) (Purchased from Pierce) according to a protocol from the manufacturer. The resin (400 μL) was washed three times with distilled water and one time with 0.1 M imidazole pH 6. After the washes, 200 uL of the imidazole solution, 32 nmol of DNA ligand 1.12.2 in water and 400 uL of 156 mM EDC in 0.1 M imidazole pH 6, were added. The reaction was incubated for 3 h at room temperature with rotation. The concentration of DNA remaining in the supernatant was estimated through acrylamide gel electrophoresis. The resin was washed several times with binding buffer BB (10 mM TRIS pH 8.5; 120 mM NaCl, 5 mM KCl, 20 mM CaCl₂) and 100 μL aliquots of resin were packed in a column made with a pipette tip (Sorensen barrier tips). To test that the conjugated DNA ligand was active, 1 mL of a 100 nM OTA solution was passed through the column and the presence of OTA in the solution was measured by its fluorescence before and after passage through the column.

To determine OTA concentration within wheat grain, ten grams of finely ground wheat grain obtained from Romer laboratories and certified to contain 2.7±1.0 ug/Kg of OTA were mixed with 40 mL of 60% methanol in distilled water in a 50 mL Falcon tube. The flour was mixed well with the solvent and shaken by hand for 5 min. The tube was centrifuged in a Centra CL2 centrifuge (Thermo ECL) at g×2500 for 5 min. A 3.5 mL aliquot of the grain extract was mixed with 10.5 mL of BB, whereupon a translucent suspension was observed. This mixture was spun for 5 min at g×12000. Experiments were performed demonstrating that OTA was not co-precipitated with this suspension. The supernatant, 12 mL, was run through the affinity column with the aid of a syringe at a rate of 1 mL per min. The column was washed with 6 mL of BB and OTA was eluted from the column with 2 mL of 20% methanol in TE buffer (10 mM TRIS, 1 mM EDTA) pH 9. The concentration of OTA in the eluate was determined fluorometrically, by comparing the intrinsic fluorescence of OTA with that of a calibration curve made in the same buffer (λexc=375 nm and λem=430 nm). Using the concentration of OTA in the eluates from the DNA ligand affinity column we calculated the quantity of OTA in the original wheat sample. The analysis was replicated with four independent samples. A negative control sample of wheat grain was purchased from Sigma™ (certified reference material) that did not contain OTA. This sample was tested twice as described above.

In preliminary experiments we found that the binding affinity between OTA and the DNA ligand 1.12.2 (SEQ ID NO: 20) was not affected significantly with concentrations of methanol up to 25%. The resulting mix of extraction solvent and BB in this experiment contained 15% methanol. The fluorometric determination of OTA in the eluate generated concentrations of OTA not significantly different than the value provided by the suppliers of the standard samples (Table 5).

We also evaluated test samples of grain provided by the Canadian Grain Commission (Grain Research Laboratory, Winnipeg, Canada). The amount of OTA in these samples was determined by researchers at the Grain Research Laboratory using Vicam antibody based affinity columns followed by fluorescence detection in a high performance liquid chromatography apparatus. The values obtained by the Grain Research Laboratory were not shared with the inventors until after we shared our results with them. These samples were treated as described above for the reference material, and were analyzed in replicate. The results from this analysis also did not differ in a statistically significant manner α=0.05 (Table 5). Clearly the inventors have developed a novel approach for the determination of mycotoxins in agricultural products through the use of a DNA ligand based affinity column. This novel approach has a significant advantage over similar antibody based approaches in that it is requires much less dilution of the sample extract and thus provides a greater level of sensitivity, and reliability.

### Example 8

### Tolerance of DNA Ligand Based Affinity Column to Various Levels of Organic Solvents

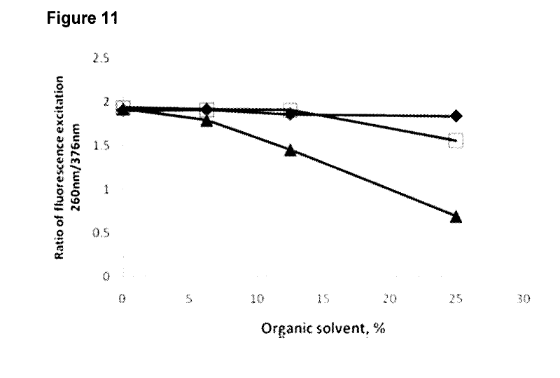

Approved methods for the extraction of mycotoxins from agricultural products all involve the use of organic solvents such as methanol or acetonitrile. Dimethyl sulfoxide (DMSO) is also frequently used to accelerate solubility of mycotoxins. The inventors of this invention realized that there was a need for a detection system that would tolerate higher levels of organic solvents such as these for use in the detection of mycotoxins. The effect of the organic solvent on the DNA ligand/OTA interaction was evaluated by measuring the shift in fluorescence spectra exhibited by OTA upon binding to the DNA ligand OTA1.12 (SEQ ID NO: 1) with a constant amount of OTA and DNA ligand, but varying levels of organic solvent (FIG. 11). The ratio of fluorescence between the two peaks is a measure of the binding between the DNA ligand and OTA. It is clear from these results that up to 25% methanol in water does not significantly inhibit DNA ligand binding activity for this mycotoxin. Similarily a level of 10% ethanol, does not significantly affect the binding of the DNA ligand disclosed in this invention and the mycotoxin OTA. This level of tolerance to ethanol is of commercial value as this facilitates the direct application of the DNA ligand for the determination of OTA in alcoholic drinks such as beer and wine. Tolerance of the DNA ligand to acetonitrile is adequate to a level of 5%. This is of value in terms of reducing dilutions of extracted solvents, thereby maintaining target concentration at a higher level, and increasing the robustness and sensitivity of the test.

### Example 9

### Enhancement of DNA Ligand Binding to Ochratoxin A Through the Substitution of Calcium for Magnesium in the Binding Buffer

The binding assays were performed in accordance with Binding Assays of Example 1 using a basic composition of the selection or binding buffer comprising of 10 mM HEPES pH 7.0, 120 mM NaCl and 5 mM KCl plus a divalent magnesium cation or a divalent calcium cation.

As shown in Table 6, binding of OTA to the DNA ligands of the present invention depended on the presence of divalent cations in the binding/selection buffer and no binding was detected when the basic composition was used without the presence of a divalent cation.

TABLE 1
Binding of DNA ligands and lack of binding
of a random oligonucleotide to OTA.

| | k_{D} (uM) | +/− |
|---|---|---|
| OTA1.12 (SEQ ID NO: 1) | 0.36 | 1.38 |
| OTA1.13 (SEQ ID NO: 2) | 6.74 | 0.44 |
| OTA1.14 (SEQ ID NO: 3) | 0.99 | 0.77 |
| OTA2.2 (SEQ ID NO: 4) | 1.59 | 0.08 |
| OTA2.3 (SEQ ID NO: 5) | 1.72 | 0.49 |
| OTA2.4 (SEQ ID NO: 6) | 19.53 | 13.26 |
| OTA2.6 (SEQ ID NO: 7) | 7.11 | 1.04 |
| OTA2.9 (SEQ ID NO: 8) | 0.96 | 0.09 |
| OTA2.10 (SEQ ID NO: 9) | 4.29 | 1.37 |
| OTA2.11 (SEQ ID NO: 10) | 2.53 | 0.45 |
| OTA2.12 (SEQ ID NO: 11) | 0.97 | 0.47 |
| OTA2.13 (SEQ ID NO: 12) | 1.92 | 0.04 |
| SR¹ (SEQ ID NO: 16) | 203.44 | 77.98 |

¹SR = Sulforhodamide DNA Ligand

TABLE 2
Relationship between consensus
sequence and binding of DNA ligands of this invention to OTA

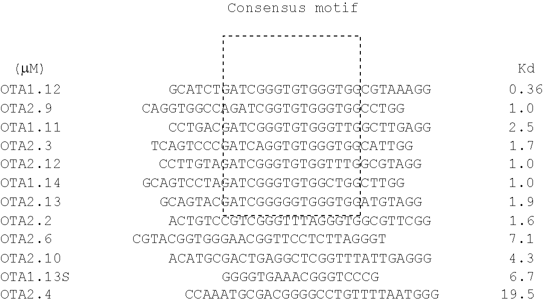

TABLE 3
Comparison of binding affinities for oligonucleotides developed
through truncations of the OTA1.12 sequence (SEQ ID NO: 1)

| | kD (uM) | +/− |
|---|---|---|
| OTA1.12 (SEQ ID NO: 1) | 1.71 | 0.64 |
| OTA1.13S (SEQ ID NO: 17) | No binding | |
| OTA1.12.1 (SEQ ID NO: 18) | 1.74 | 0.03 |
| OTA1.12.2 (SEQ ID NO: 19) | 1.3 | 0.2 |
| OTA1.12.3 (SEQ ID NO: 20) | 403.8 | n.a. |
| OTA1.12.4 (SEQ ID NO: 21) | 466.9 | 356 |
| OTA1.12.5 (SEQ ID NO: 22) | 830 | 300 |
| OTA1.12.6 (SEQ ID NO: 23) | No binding | |

n.a. = not available

TABLE 4
Description of variables used in
OTA/DNA ligand selection strategy

| | | Positive selection | |
|---|---|---|---|
| Selection | Negative | incubation time | Counter |
| Cycle | selection | (min) | selection |
| 1 | yes | 60 | No |
| 2 | No | 60 | No |
| 3 | No | 60 | yes |
| 4 | No | 60 | yes |
| 5 | No | 60 | yes |
| 6 | No | 60 | yes |
| 7 | No | 60 | yes |
| 8 | No | 60 | yes |
| 9 | No | 10 | yes |
| 10 | No | 5 | yes |
| 11 | yes | 1 | yes |
| 12 | yes | 1 | yes |
| 13 | No | 1 | yes |

TABLE 5
Analytical results derived through the use of
a DNA ligand based affinity column for OTA

| | Certified | Concentration determined with |
|---|---|---|
| | concentration, | a DNA ligand affinity column, |
| Sample | μg/Kg | μg/Kg |
| Sigma blank | <0.6 | −0.4 ± 0.8 |
| Romer labs | 2.7 ± 1.0 | 2.2 ± 0.4 |
| GRL sample #1 | 1.8 ± 0.6 | 2.3 ± 1.1 |
| GRL sample #2 | 5.0 ± 1.5 | 6.8 ± 3.4 |
| GRL sample #3 | 7.4 ± 1.9 | 9.5 ± 2.0 |
| GRL sample #4 | 61.9 ± 7.5 | 56.1 ± 9.0 |
| GRL sample #5 | <1.0 | 0.47 ± 0.09 |

TABLE 6
Effect of magnesium and calcium concentration on the binding
affinity between OTA and DNA ligand 1.12.2 (SEQ ID NO: 20)
in Binding Buffer with and without divalent cations

Kd, nM

| Concentration, mM | Mg2+ | Ca2+ |
|---|---|---|
| 0 | NB | NB |
| 5 | 197 ± 74 | 112 ± 12 |
| 10 | 148 ± 8 | 54 ± 8 |
| 20 | 118 ± 46 | 49 ± 3 |

1. A DNA ligand characterized in that said DNA ligand binds to a mycotoxin.

2. The DNA ligand of claim 1 characterized in that said mycotoxin is selected from the group consisting of: deoxynivalenol, zearalenone, T2-toxin, aflatoxin B1 , fumosins, patulin, and ochratoxin A.

3. (canceled)

4. The DNA ligand of claim 1 characterized in that said DNA ligand comprises a nucleotide sequence selected from the group consisting of SEQ ID NOs: 1 to 13, 18 to 22, or a functional analogue or variant thereof.

5-7. (canceled)

8. A method for detecting a mycotoxin in a sample characterized in that the method comprises:
(a) contacting said sample to a DNA ligand that binds to said mycotoxin under conditions wherein a mycotoxin/DNA ligand complex is formed if said mycotoxin is present in the sample; and
(b) determining whether said mycotoxin/DNA ligand complex is formed, thereby detecting the mycotoxin in the sample.

9-12. (canceled)

13. The method of claim 8 characterized in that formation of said complex is determined by fluorescence, high performance liquid chromatography, mass spectrometry of the mycotoxin, the use of fluorescence in combination with quenchers or fluorescence polarization.

14. The method of claim 8 characterized in that said conditions wherein a mycotoxin/DNA ligand is formed comprises using a composition having a cation for enhancing the binding of said DNA ligand to said mycotoxin, wherein said cation is selected from the group consisting of: sodium, potassium, calcium, scandium, titanium, vanadium, chromium, manganese, iron, cobalt, nickel, copper and zinc.

15. The method of claim 8 characterized in that said sample is an agriculture product.

16-17. (canceled)

18. The method for detecting the presence or absence of a mycotoxin in a sample of claim 8 characterized in that said mycotoxin is selected from the group consisting of: deoxynivalenol, zearalenone, T2-toxin, aflatoxin B1, fumosins, patulin and ochratoxin A.

19. (canceled)

20. The method of claim 8 characterized in that said DNA ligand comprises a nucleotide sequence selected from the group consisting of: SEQ ID NOs: 1 to 13, 18 to 22 or a functional analogue thereof or a variant thereof.

21-22. (canceled)

23. A method for determining the concentration of a mycotoxin in a sample characterized in that said method comprises:
(a) contacting said sample to a DNA ligand that binds to said mycotoxin under conditions wherein a mycotoxin/DNA ligand complex is formed if said mycotoxin is present in the sample; and
(b) quantitatively detecting the mycotoxin/DNA ligand complex, thereby determining the concentration of the mycotoxin in the sample.

24-25. (canceled)

26. The method of claim 23 characterized in that step (b) comprises releasing said mycotoxin from the mycotoxin/DNA complex and using detection means to measure the amount of said released mycotoxin.

27. The method of claim 23 characterized in that the detection means is selected from the group consisting of: fluorescence, high performance liquid chromatography, mass spectrometry of the mycotoxin, the use of fluorescence in combination with quenchers and fluorescence polarization.

28. The method of claim 23 characterized in that said conditions wherein a mycotoxin/DNA ligand is formed comprises using a composition having a cation for enhancing the binding of said DNA ligand to said mycotoxin, wherein said cation is selected from the group consisting of: sodium, potassium, calcium, scandium, titanium, vanadium, chromium, manganese, iron, cobalt, nickel, copper and zinc.

29. The method of claim 23 characterized in that said sample is an agricultural product.

30-31. (canceled)

32. The method of claim 23 characterized in that said mycotoxin is selected from the group consisting of: deoxynivalenol, zearalenone, T2-toxin, aflatoxin B1, fumosins, patulin and ochratoxin A.

33. (canceled)

34. The method of claim 23 characterized in that said DNA ligand comprises a nucleotide sequence selected from the group consisting of: SEQ ID NOs: 1 to 13, 18 to 22 or a functional analogue thereof or a functional variant thereof.

35-36. (canceled)

37. A method for removing or reducing the level of a mycotoxin present in a sample characterized in that said method comprises:
(a) contacting said sample with a DNA ligand that binds to said mycotoxin under conditions wherein a mycotoxin/DNA ligand complex is formed if said mycotoxin is present in the sample; and
(b) removing the mycotoxin/DNA ligand complex from the sample.

38-39. (canceled)

40. The method of claim 37 characterized in that said conditions wherein a mycotoxin/DNA ligand is formed comprises using a composition having a cation for enhancing the binding of said DNA ligand to said mycotoxin wherein said cation is selected from the group consisting of: sodium, potassium, calcium, scandium, titanium, vanadium, chromium, manganese, iron, cobalt, nickel, copper and zinc.

41. The method of claim 37 characterized in that said sample is an agricultural product.

42-43. (canceled)

44. The method of claim 37 characterized in that said mycotoxin is selected from the group consisting of: deoxynivalenol, zearalenone, T2-toxin, aflatoxin B1, fumosins, patulin and ochratoxin A.

45. (canceled)

46. The method of claim 37 characterized in that said DNA ligand comprises a nucleotide sequence selected from the group consisting of: SEQ ID NOs: 1 to 13, 18 to 22 or a functional analogue thereof or a functional variant thereof.

47-67. (canceled)

68. A composition for enhancing the affinity of a nucleic acid ligand to a target of said nucleic acid ligand, comprising a cation, wherein said cation is selected from the group consisting of: sodium, potassium, calcium, scandium, titanium, vanadium, chromium, manganese, iron, cobalt, nickel, copper and zinc.

69-71. (canceled)

72. A method for the identification of DNA ligands that bind to a mycotoxin of interest characterized in that the method comprises:
(a) contacting a random library of single stranded DNA sequences to the mycotoxin of interest under conditions wherein a mycotoxin/DNA ligand complex is formed between the DNA ligands within the random library and the immobilized mycotoxin of interest; and
(b) releasing the single stranded DNA sequences from the mycotoxin/DNA complexes, wherein said released DNA sequences are the DNA ligands that bind to the mycotoxin of interest.

73. The method of claim 72 characterized in that said contacting step is done in the presence of an organic solvent.

74. The method of claim 72 characterized in that said method further comprises a step for selecting for DNA ligand specificity for the mycotoxin of interest, wherein said step comprises exposing the mycotoxin/DNA complex to one or more molecules having substantially similar chemical structures to the mycotoxin of interest.

75. The method of claim 73 characterized in that the organic solvent is selected from the group of organic solvents consisting of: a solvent having up to 2.5% dimethyl sulfoxide, a solvent having up to about 25% methanol, a solvent having up to about 10% ethanol, and a solvent having up to about 5% acetonitrile.

76. The method of claim 72 characterized in that said mycotoxin is selected from the group consisting of: deoxynivalenol, zearalenone, T2-toxin, aflatoxin B1, fumosins, patulin and ochratoxin A.

77. The method of claim 72 characterized in that said mycotoxin is ochratoxin A and wherein said DNA ligand comprises a nucleotide sequence selected from the group consisting of SEQ ID NOs: 1 to 13, 18 to 22 or a functional analogue thereof or variant thereof.

78. The method of claim 72 characterized in that said conditions wherein a mycotoxin/DNA ligand complex is formed comprises using a composition having a cation, wherein said cation is selected from the group consisting of: sodium, potassium, calcium, scandium, titanium, vanadium, chromium, manganese, iron, cobalt, nickel, copper and zinc.

79. A DNA sequence characterized in that said DNA sequence comprises at least one nucleotide sequence selected from the group consisting of: SEQ ID NOs: 1 to 13 and SEQ ID NOs: 17 to 23.
